(19) United States
(12) Patent Application Publication
(10) Pub. No.: US 2007/0194046 A1
(43) Pub. Date: Aug. 23, 2007

(54) DEVICE FOR DISPENSING A FLOWABLE SUBSTANCE

(76) Inventor: Alberto C. Sogaro, Kronberg (DE)

Correspondence Address: QUARLES & BRADY LLP, 411 E. WISCONSIN AVENUE, SUITE 2040, MILWAUKEE, WI, 53202-4497, US

(21) Appl. No.: 11/676,385

(22) Filed: Feb. 19, 2007

(30) Foreign Application Priority Data
Feb. 23, 2006 (DE) 20 2006 002 927.3

(51) Int. Cl. A61M 5/00; B65D 35/00; B67D 5/60

(52) U.S. Cl. 222/107; 222/145.5; 604/191

(57) ABSTRACT

A device for dispensing a flowable substance includes a tubular reservoir (12) forming a reservoir chamber (14), which is closed on one end and changes into a neck section (16) with a discharge opening (18) on the end facing away from the closed end. A closure (20) is molded onto the neck section (16), which closes the discharge opening (18) and can be separated from the neck section (16) in order to activate the device. The neck section (16) is surrounded by an application device (26). The closure (20) is developed in form of a mandrel which penetrates the application device (26) in such a manner that it projects beyond the application device (26).

### CROSS REFERENCES TO RELATED APPLICATIONS

This application claims the priority benefit of German Utility Model No. 20 2006 002 927.3 filed on Feb. 23, 2006

### STATEMENT REGARDING FEDERALLY SPONSORED RESEARCH

Not Applicable.

### DESCRIPTION OF THE BACKGROUND ART

The invention relates to a device for dispensing a flowable substance with a tubular reservoir forming a reservoir chamber, which is closed on one end and changes into a neck section with a discharge opening on the end facing away from the closed end. A closure is molded onto the neck section, which closes the discharge opening and can be separated from the neck section in order to activate the device.

Such devices are of the prior art and are designed for example in the form of disposable dispensing devices for a liquid, for example a liquid medication. Disposable dispensing devices should on the one hand be able to be manufactured economically and on the other be able to be operated reliably and easily. Moreover, a dispensing device for medical applications should be particularly designed so that the flowable substance can be applied as precisely as possible and without loss.

### SUMMARY OF THE INVENTION

An object of the present invention is to create a device of the type mentioned in the introduction that can be easily activated and which can be used for the precise application of the respective flowable substance. This is accomplished by a dispensing device having the neck section surrounded by an application device with a mandrel-shaped closure (20) penetrating the application device in such a manner that it projects beyond the application device.

The present invention provides a device for dispensing a flowable substance includes a tubular reservoir which has a reservoir chamber, which is closed on one end and changes into a neck section with a discharge opening on the end facing away from the closed end. A closure is molded onto the neck section, which closes the discharge opening and can be separated from the neck section in order to activate the device. The neck section is surrounded by an application device in which the closure is mandrel-shaped and penetrates the application device in such a manner that it projects beyond the application device.

The design of the device incorporating the invention makes it possible to transfer a flowable substance, after separating the closure from the neck section, from the reservoir chamber of the reservoir to the application device and/or load the flowable substance into it without loss, since the discharge opening leads into the area of the application device. The application device is developed preferably essentially annular and constitutes a brush, a sponge, a cotton body or a fluffy body, for example.

In order to protect the application device in the storage condition, in a preferred embodiment, the closure is provided with a cap for the application device. The cap, for example, is essentially a cup-shaped design. It preferably has a height which corresponds essentially to the longitudinal dimension of the application device.

The protective cap for the application device can have a female thread, which engages with the male thread of the reservoir so that when twisting the cap, the closure fixed to the cap can be unfastened from the neck section in a predetermined manner. It is naturally also conceivable to provide a male thread on the protective cap and a female thread on the reservoir. In this case, the protective cap inserts into the reservoir.

Due to technical manufacturing reasons, it may be necessary to separately manufacture the cap and the mandrel-shaped closure, which can be manufactured in one piece with the reservoir and the neck section. In this case, the cap can be engaged on the free end of the mandrel-shaped closure or may also be bonded onto the free end of the mandrel-shaped closure.

In an alternative embodiment, a gripping strip can be designed on the mandrel-shaped closure in order to facilitate the activation on its free end which protrudes beyond the application device. This can be provided with fluting or the like.

Preferably, the reservoir of the device according to the invention is an elastic, ductile plastic part, so that by applying lateral manual pressure, the flowable substance contained in the reservoir chamber can be delivered to the application device via the discharge opening. However, alternatively it is also conceivable to manufacture the reservoir from rigid plastic material, such as COC, in which case the mandrel-shaped closure can be broken off from the neck section, which is likewise preferably developed from rigid material, in order to activate the device. A special embodiment of the device according to the invention can furthermore have a circumferential corrugations providing a bellows-like designed area on the closed end section of the reservoir, so that this area serves as a pump unit to discharge the flowable substance from the reservoir chamber of the reservoir. The reservoir chamber can in addition be provided with a graduated scale in order to exactly determine the quantity of the flowable substance to be discharged.

It is conceivable to manufacture the neck section and the reservoir from different materials. In this case it is useful to design the neck section with the discharge opening as an insert of the reservoir.

The device as according to the invention can also be designed to discharge a multicomponent system in which the components must be mixed during application. In this case, the reservoir has two or even more chambers. Each of the chambers can be provided with its own discharge opening, which is closed with a common closure according to the invention. Then, the components would only be mixed outside of the reservoir. It is also conceivable to separate the chambers from each other by means of a detachable sealed joint or such like, where the sealed joint can be detached by manual pressure prior to detaching the closure, so that the components stored within the individual chambers can still mix in the reservoir.

Additional advantages and advantageous embodiments of the subject matter of the invention can be found in the description, in the drawing, and in the Claims.

### BRIEF SUMMARY OF THE DRAWINGS

Two embodiments of the device according to the invention are shown in the drawing in a simplified schematic representation, which are explained in detail in the following description.

FIG. 1 shows a longitudinal section through a first embodiment of a discharge device designed according to the invention; and

FIG. 2 shows a longitudinal section through a second embodiment of a discharge device designed according to the invention.

### DETAILED DESCRIPTION OF THE PREFERRED EMBODIMENTS

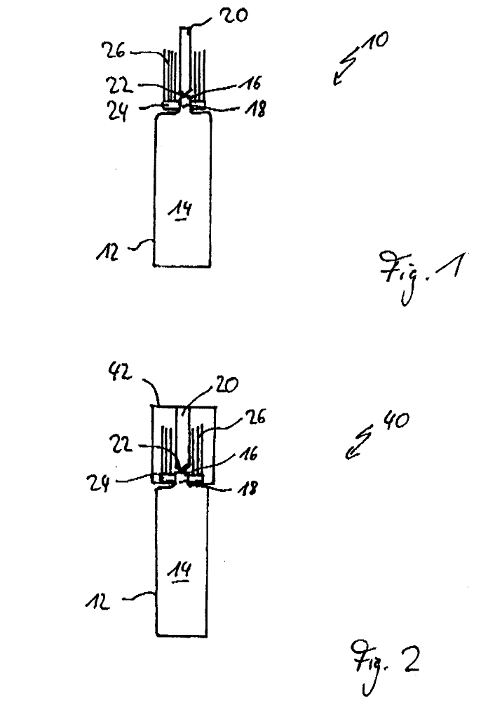

FIG. 1 shows a discharge device 10, which can be used for the application of a flowable substance used in the medical field, such as a medication or a tissue adhesive. The discharge device 10 has a reservoir 12 which is an elastic, ductile plastic part and which contains a reservoir chamber 14 for holding the flowable substance. The reservoir 12 is closed on the end shown in the bottom of FIG. 1 and changes into a neck section 16 with a reduced diameter shown in the top of FIG. 1, which has a channel 18 forming a discharge opening. The channel 18 and/or the discharge opening is closed by a mandrel-shaped, rod-like closure 20, whose axis is aligned with the axis of the neck section 16, and which is molded, particularly by injection molding, onto the neck section 16 of the reservoir 12 by means of a designated separation point 22 with a reduced cross-section.

The neck section 16 is furthermore surrounded by a support ring 24 which is connected to the neck section 16 by means of press fit and which serves to support an application device 26, which is designed as a brush in the present case, whose bristles are bonded onto the support ring 24.

The mandrel-shaped closure 20 penetrates the application device 26 in such a manner that it projects beyond the end of the application device 26 facing away from the support ring 24 and can thus be easily gripped by a user for activating the device 10, i.e. for opening the discharge opening 18, and can thus be separated and/or unfastened in the area of the designated separation point 22. In order to apply the liquid contained in the reservoir chamber 14, the user applies lateral pressure manually to the reservoir 12, so that the applicator 26 is loaded with the liquid contained in the reservoir chamber 14 via the discharge opening 18, and the liquid can be applied with the applicator 26 onto which it has been loaded.

The embodiment of a discharge device 40 illustrated in FIG. 2 differs from the embodiment shown in FIG. 1 to the extent that the discharge device 40 has a cup-shaped cap 42, which on the one hand protects the applicator 26, designed as a brush, and on the other facilitates the separation of the closure 20. For this purpose, the cap 42 engages in the area of its base 44 with the free end of closure 20 that faces away from the reservoir 12. In other respects, the discharge device 40 corresponds to the embodiment shown in FIG. 1.

In order to open the discharge opening 18 of the discharge device 40, a user grips cap 42, which can be provided with fluted areas along its periphery, preferably on the side, turns it and thereby detaches the closure 20 from the neck section 16 of the reservoir 12.

I claim:

1. A device for dispensing a flowable substance, said device comprising;
a tubular reservoir (12) forming a reservoir chamber (14), which is closed on a first end and changes into a neck section (16) with a discharge opening (18) on a second end facing away from the closed first end;
a mandrel-shaped closure (20) is molded onto the neck section (16), which closes the discharge opening (18) and can be separated from the neck section (16) in order to activate the device; and
an application device (26) surrounding the neck section (16) and penetrated by the closure (20), wherein the closure (20) projects beyond the application device (26).

2. The device as in claim 1, wherein the closure (20) is provided with a cap (42) for the application device (26).

3. The device as in claim 2, wherein the cap (42) engages into the closure (20).

4. The device as in claim 1, wherein a gripper bottle has been developed on the closure.

5. The device as in claim 1, wherein the application device (26) comprises brush bristles.

6. The device as in claim 1, wherein the application device (26) comprises a support ring (24), which surrounds the neck section (16).

7. The device as in claim 1, wherein the reservoir (12) is an elastic, ductile plastic part.

8. The device as in claim 1, wherein the reservoir includes a bellows in proximate the closed end.

9. The device as in claim 1, wherein the reservoir is graduated.

10. The device as in claim 1, wherein the neck section with the discharge opening is developed as an insert of the reservoir.
